(19) United States
(12) Patent Application Publication
(10) Pub. No.: US 2022/0135921 A1
(43) Pub. Date: May 5, 2022

(54) METHODS AND APPARATUS FOR CELL DEVELOPMENT

(71) Applicant: APPLIED MATERIALS, INC., Santa Clara, CA (US)

(72) Inventors: Mukhles SOWWAN, Cupertino, CA (US); Samer BANNA, San Jose, CA (US)

(21) Appl. No.: 17/307,439

(22) Filed: May 4, 2021

Related U.S. Application Data
(60) Provisional application No. 63/107,262, filed on Oct. 29, 2020.

(51) Int. Cl. C12M 3/00; C12M 1/12; C12M 1/00; C12N 5/00; C12N 5/0783

(52) U.S. Cl. CPC C12M 21/08; C12M 25/14; C12M 25/02; C12N 2513/00; C12N 5/0062; C12N 5/0636; C12M 23/34

(57) ABSTRACT

Embodiments of methods and apparatus for T-cell activation, T-cell transfection, and T-cell expansion are provided herein. For example, the apparatus includes a pump connected to a circulation path and configured to circulate cells suspended in a fluid to and from a container connected to the circulation path, the circulation path comprising a 3D printed blood vessel bed comprising in order of cell flow an artery-scaled vessel, an arteriole-scaled vessel, a capillary-scaled vessel, a venule-scaled vessel, and a vein-scaled vessel.

### CROSS-REFERENCE TO RELATED APPLICATIONS

The present application claims the benefit of and priority to U.S. Provisional Patent Application Ser. No. 63/107,262, which was filed on Oct. 29, 2020, the entire contents of which is incorporated herein by reference.

### FIELD

Embodiments of the present disclosure generally relate to a methods and apparatus for cell development, and more particularly, to methods and apparatus that are configured to mimic an in vivo environment during cell expansion.

### BACKGROUND

Cell expansion systems (CESs) that are used to expand cells (e.g., T cells, Stem cells, bone marrow cells, etc.) for use with one or more cell therapies (e.g., chimeric antigen receptor T cell (CAR-T cell) therapy) are known. For example, CESs can include one or more compartments for growing the cells, such as a cell growth chamber, e.g., a bioreactor. Conventional CESs, however, do not provide macro and micro scale cell processing in a single platform, are not configured to mimic an in vivo environment (e.g., a human cardiovascular system including heart, capillaries, veins, arteries, etc.), and do not provide point of care treatment in a cost-effective manner.

### SUMMARY

Methods and apparatus for T-cell activation, T-cell transfection, and T-cell expansion are provided herein. For example, an apparatus can include a pump connected to a circulation path and configured to circulate cells suspended in a fluid to and from a container connected to the circulation path, the circulation path comprising a 3D printed blood vessel bed comprising in order of cell flow an artery-scaled vessel, an arteriole-scaled vessel, a capillary-scaled vessel, a venule-scaled vessel, and a vein-scaled vessel.

In at least some embodiments a method for point of care treatment of a patient includes sterilely receiving patient T-cells into a container connected to a circulation path and a pump that is configured circulate the patient T-cells to and from the container, the circulation path comprising a 3D printed blood vessel bed comprising in order of cell flow an artery-scaled vessel, an arteriole-scaled vessel, a capillary-scaled vessel, a venule-scaled vessel, and a vein-scaled vessel, expanding the patient T-cells while flowing a constant portion of the patient T-cells through the 3D printed blood vessel bed, and sterilely withdrawing expanded patient T-cells in the container for administration back into the patient.

In at least some embodiments a method of activation, transfection, and expansion of T-cells includes receiving T-cells into a container connected to a circulation path and a pump that is configured circulate the T-cells to and from the container, the circulation path comprising a single use 3D printed blood vessel bed comprising in order of cell flow an artery-scaled vessel, an arteriole-scaled vessel, a capillary-scaled vessel, a venule-scaled vessel, and a vein-scaled vessel, flowing a constant portion of the T-cells through the single use 3D printed blood vessel bed, and withdrawing at least one of activated, transfected, or expanded T-cells in the container.

Other and further embodiments of the present disclosure are described below.

### BRIEF DESCRIPTION OF THE DRAWINGS

Embodiments of the present disclosure, briefly summarized above and discussed in greater detail below, can be understood by reference to the illustrative embodiments of the disclosure depicted in the appended drawings. However, the appended drawings illustrate only typical embodiments of the disclosure and are therefore not to be considered limiting of scope, for the disclosure may admit to other equally effective embodiments.

FIG. 1 is a diagram of a system for T-cell activation, transfection, genetic modification, and expansion, in accordance with at least some embodiments of the present disclosure.

FIG. 2 is a diagram of the indicated area of detail 2 of FIG. 1, in accordance with at least some embodiments of the present disclosure.

FIG. 3 is a diagram of the indicated area of detail 3 of FIG. 2, in accordance with at least some embodiments of the present disclosure.

FIG. 4 is a flowchart of a method for point of care treatment of a patient, in accordance with at least some embodiments of the present disclosure.

FIG. 5 is a flowchart of a method of activation, transfection, and expansion of T-cells, in accordance with at least some embodiments of the present disclosure.

To facilitate understanding, identical reference numerals have been used, where possible, to designate identical elements that are common to the figures. The figures are not drawn to scale and may be simplified for clarity. Elements and features of one embodiment may be beneficially incorporated in other embodiments without further recitation.

### DETAILED DESCRIPTION

Embodiments of a methods and apparatus that are configured to mimic an in vivo environment during cell expansion are provided herein. For example, the apparatus can be configured as a closed loop platform (e.g., a bioreactor) for activation, transfection, genetic modification, and expansion of T cells, stem cells, bone marrow cells, or other types of cells used during a cell manufacturing process, e.g., a CAR-T cell manufacturing process. In at least some embodiments, the methods and apparatus described herein are configured to mimic various aspects of a human cardiovascular system (e.g., heart, capillaries, veins, arteries, etc.) and provide macro and micro scale cell processing in a single platform, which, in turn, can provide point of care treatment in a relatively cost-effective manner.

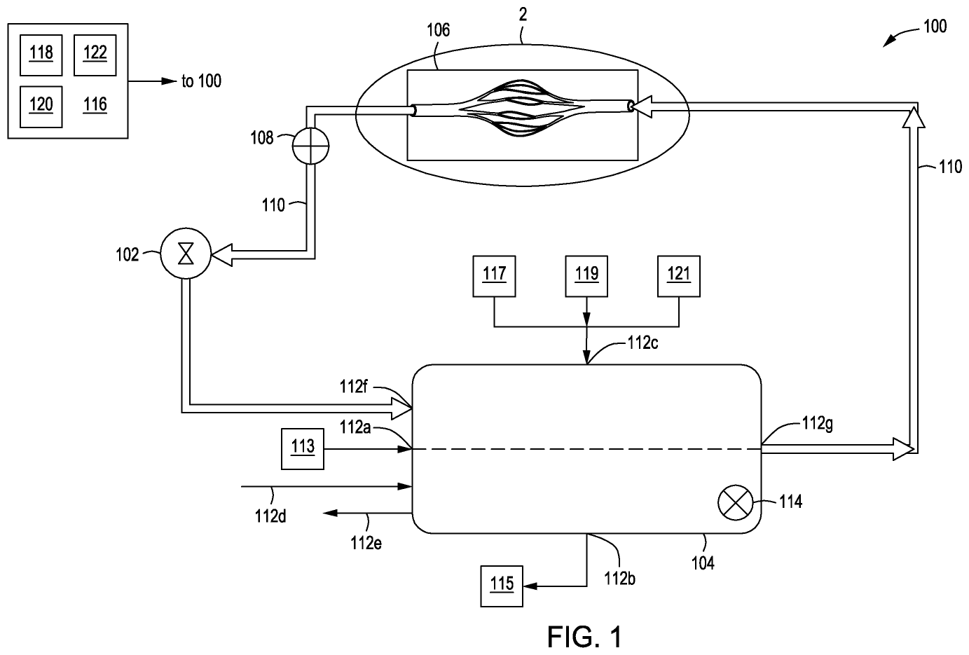

FIG. 1 is a diagram of a system 100 for T-cell activation, T-cell transfection, T-cell genetic modification, and T-cell expansion in accordance with at least some embodiments of the present disclosure.

The system 100 includes a pump 102, a container 104 (e.g., a bioreactor), a 3D printed blood vessel bed 106, one or more sensors 108, and a circulation path 110 (e.g., biocompatible, single use, and disposable tubing) that is configured to connect to the pump 102, the container 104, the 3D printed blood vessel bed 106, and the one or more sensors 108 via welding in a sterile welding operation or using a suitable connector. The system 100 is configured for activation, genetic modification, transfection, and expansion of one or more cells. For example, the cells can be T cells, stem cells, bone marrow cells, or other types of cells. In at least some embodiments, the cells can be T-cells used during a cell manufacturing process, e.g., a CAR-T cell manufacturing process, which can then be used for point of care treatment of a patient, as will be described in greater detail below.

Continuing with reference to the pump 102 is connected to the circulation path 110 and configured to circulate T-cells, which can be suspended in a fluid, to and from the container 104 via the circulation path. The pump 102 can be any type of pump that is capable of circulating the T-cells. Suitable pumps can include, but are not limited to, peristaltic pumps, single speed or variable speed, AC or DC, programmable/non-programmable. For example, in at least some embodiments, the pump 102 is a peristaltic pump. A peristaltic pump, also commonly known as a roller pump, is a type of positive displacement pump used for pumping a variety of fluids. The fluid is contained within a flexible tube fitted inside a circular pump casing (though linear peristaltic pumps have been made). The pump 102 (and the other components of the system 100) is configured to connect to the circulation path 110 via welding in a sterile welding operation or thorough an appropriate connector. For example, in at least some embodiments, the pump 102 comprises an input port and output port (not shown) that connect to the circulation path 110 via welding (not shown).

The container 104 can be made from one or more materials suitable for use with bioreactors. For example, in at least some embodiments, the container 104 can be made from a biocompatible plastic (e.g., a polymeric material) capable of maintaining a sterile environment within an inner volume of the container 104. The container 104 includes multiple ports 112a-112g each of which are configured to connect to corresponding one a gas supply 113 (port 112a), connect to a waste line 115 (port 112b), connect to a cell suspension apparatus 117, connect to a reagent apparatus 119, or connect to a media apparatus 121 or in line monitoring devices and sensors. In the illustrated embodiment, the cell suspension apparatus 117, the reagent apparatus 119, and the media apparatus 121 (or in line monitoring devices and sensors) are configured to share a port 112c. Alternatively, the cell suspension apparatus 117, the reagent apparatus 119, and the media apparatus 121 can be connected to respective ports. Additionally, an input port 112d and an output port 112e can be provided and are configured to receive/withdraw patient T-cells, as will be described in greater detail below. The container 104 includes ingress and egress ports 112f and 112g that are configured to connect to the circulation path 110 via welding in a sterile welding operation or through an appropriate connector.

One or more valves (not shown) can be coupled to the ports 112a-112f and can be configured to control fluid flow in or out of the container 104. Additionally, a temperature sensor 114 is disposed within the container 104 and is configured to measure temperature of fluid (e.g., from about 0° C. to about 50° C.) within the container 104 such as platinum resistance thermometer. In at least some embodiments, one or more heating/cooling apparatus (e.g., resistive heaters, electrodes, coils, closed-loop channels configured to provide one or more fluids configured to heat/chill the fluid in the container 104).

The circulation path 110 can be made from one or more suitable materials capable of maintaining a sterile environment within an inner volume of the circulation path 110. For example, in at least some embodiments, the circulation path can be made from thermoplastic elastomer such as platinum-cured silicone.

The one or more sensors 108 (e.g., a smart sensor or multi sensor) are configured to at least one to measure a glucose, lactate, glutamine, glutamate, pH, CO₂ or dissolved O level in the fluid, a pressure within the circulation path 110, a flow rate of the fluid through the circulation path 110, or a temperature of the fluid within the circulation path 110. In at least some embodiments, the one or more sensors 108 can be disposed within the circulation path 110 between the pump 102 and the 3D printed blood vessel bed 106. Alternatively, individual sensors can be disposed within the circulation path 110 and configured to measure corresponding ones of the glucose, lactate, glutamine, glutamate, pH, CO₂ or dissolved O, the pressure, the velocity, and the temperature.

The system 100 includes or is in operable communication with a system controller 116 to control the operation of the system 100 during operation. The system controller 116 comprises a central processing unit (CPU) 118, a memory 120 (e.g., non-transitory computer readable storage medium), and support circuits 122 for the CPU 118 and facilitates control of the components of the system 100. The system controller 116 may be one of any form of general-purpose computer processor that can be used in an industrial setting for controlling various components of the system 100 and sub-processors. The memory 120 stores software (source or object code) that may be executed or invoked to control the operation of the system 100 in the manner described herein. For example, the system controller 116 is connected to the one or more sensors 108 and the temperature sensor 114 to measure the above-described variables, e.g., glucose, lactate, glutamine, glutamate, pH, CO₂ or dissolved O pressure, velocity, temperature. The system controller 116, based on one or more measured variables, is also configured (or programmed) to control one or more components of the system 100. For example, the system controller 116 can be configured to control the pump 102 to increase or decrease a flow rate of the fluid through the circulation path 110, the gas supply 113 to supply one or more gases (e.g., oxygen, carbon dioxide air) to the container 104, valves, etc.

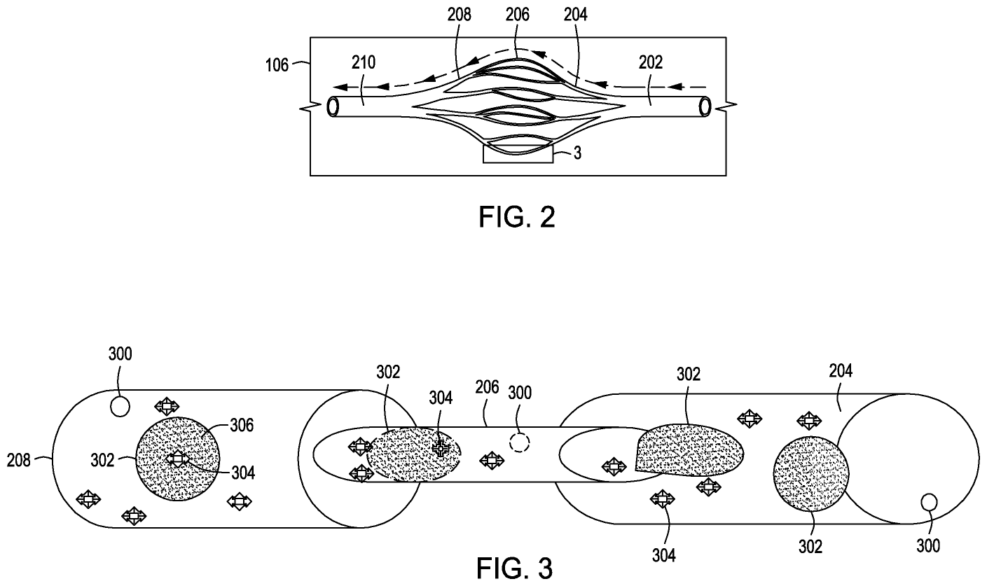

FIG. 2 is a diagram of the indicated area of detail 2 of FIG. 1 and FIG. 3 is a diagram of the indicated area of detail 3 of FIG. 2 in accordance with at least some embodiments of the present disclosure. The 3D printed blood vessel bed 106 comprises in order of cell flow an artery-scaled vessel 202, an arteriole-scaled vessel 204, a capillary-scaled vessel 206, a venule-scaled vessel 208, and a vein-scaled vessel 210. The artery-scaled vessel 202 is connected to the circulation path 110 and has an inner diameter from about 1 mm to about 2 cm. The arteriole-scaled vessel 204 is connected to the artery-scaled vessel 202 and has an inner diameter from about 20 microns to about 1 mm. The capillary-scaled vessel 206 is connected to the arteriole-scaled vessel 204 and has an inner diameter from about 5 micron to about 20 micron. The venule-scaled vessel 208 is connected to the capillary-scaled vessel 206 and has an inner diameter from about 1 mm to about 2 cm. The vein-scaled vessel 210 is connected to the venule-scaled vessel 208 and has an inner diameter from about 20 microns to about 1 mm. An interior of the artery-scaled vessel 202, the arteriole-scaled vessel 204, the capillary-scaled vessel 206, the venule-scaled vessel, and the vein-scaled vessel are smooth (e.g., without any ridges or corrugations).

At least one electrode 300 can be connected to at least one of the artery-scaled vessel 202, the arteriole-scaled vessel 204, the capillary-scaled vessel 206, the venule-scaled vessel 208, or the vein-scaled vessel 210. For illustrative purposes, the at least one electrode 300 is shown connected to the arteriole-scaled vessel 204, the capillary-scaled vessel 206, and the venule-scaled vessel 208 (FIG. 3). Under the control of the system controller 116, the at least one electrode 300 is configured to generate an electrical impulse at the at least one of the artery-scaled vessel 202, the arteriole-scaled vessel, the capillary-scaled vessel, the venule-scaled vessel, or the vein-scaled vessel. For example, in at least some embodiments, in conjunction with controlled velocity of fluid flow including the T-cells suspended therein through the capillary-scaled vessel 206, the electrical impulse generated by the at least one electrode 300 can be configured to provide cell membrane 302 disruption at the capillary-scaled vessel 206. In doing so, molecular cargo 304 can be delivered into the cytoplasm 306 of a disrupted cell membrane 302. In some embodiments, cell membrane 302 disruption can be achieved without using the electrical impulse, and just controlling the velocity of fluid flow through the artery-scaled vessel 202, the arteriole-scaled vessel 204, the capillary-scaled vessel 206, the venule-scaled vessel 208, or the vein-scaled vessel 210.

The molecular cargo 304 can be, for example, nucleic acid (RNA or DNA), CRISPR, small molecules or large molecules, which may be, or may not be, packaged, for example in a liposome or a viral capsid or envelop. The molecular cargo 304 can be presented with, for example, a transfection-enhancing substance or chemical reagent, such as calcium phosphate, cationic polymer such as DEAE-dextran or polyethylenimine, liposomes, or the like.

The 3D printed blood vessel bed can be a disposable (or single use) or sterilized and re-used. In at least some embodiments, the 3D printed blood vessel bed 106 can be disposable and can be fabricated using one or more suitable 3D printing apparatus. Alternatively, after use, the 3D printed blood vessel bed 106 can be sterilized and re-used. In at least some embodiments, the 3D printed blood vessel bed 106 is made from at least one of polymeric material, biopolymers, hydrogels, cells combined with hydrogels, or any biocompatible and 3D printing material.

The 3D printed blood vessel bed 106 is connected in line with the circulation path 110 via one or more suitable connection apparatus. For example, in at least some embodiments, one or more clamps can be used to connect the vein-scaled vessel 210 and the artery-scaled vessel 202 to the circulation path 110.

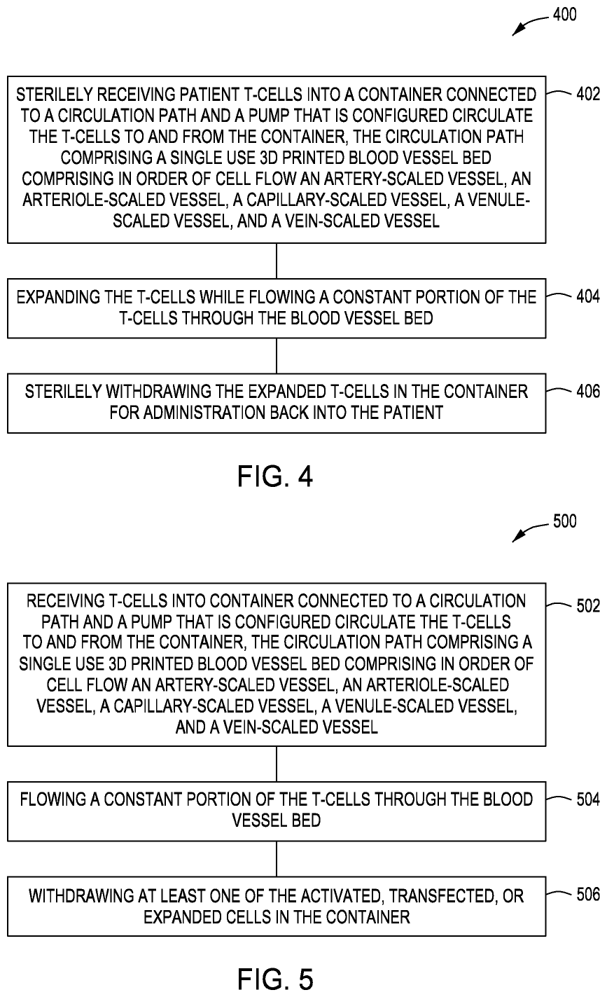

FIG. 4 is a flowchart of a method for point of care treatment of a patient in accordance with at least some embodiments of the present disclosure. For example, at 402, patient T-cells are sterilely received into a container connected to a circulation path and a pump that is configured circulate the patient T-cells to and from the container. For example, in at least some embodiments, the patient T-cells can be injected/supplied into the container via the input port 112d of the container 104. As described above, the circulation path comprises a 3D printed blood vessel bed comprising in order of cell flow an artery-scaled vessel, an arteriole-scaled vessel, a capillary-scaled vessel, a venule-scaled vessel, and a vein-scaled vessel. Next, at 404, the patient T-cells are expanded while flowing a constant portion of the T-cells through the blood vessel bed. For example, the T-cells can expand at the capillary-scaled vessel 206 of the 3D printed blood vessel bed 106. Next, at 406, the expanded patient T-cells in the container are sterilely withdrawn for administration back into the patient, e.g., using the output port 112e of the container 104. As noted above, during the method 400, the system controller 116 can be used to control one or more components of the system 100 and/or measure one or more variables associated with the system 100. For example, the system controller 116 can control a flow rate of the fluid through the circulation path, a temperature of the fluid in the container 104, etc.

FIG. 5 is a flowchart of a method of activation, transfection, and expansion of T-cells in accordance with at least some embodiments of the present disclosure. For example, at 502, T-cells are received into a container connected to a circulation path and a pump that is configured circulate the T-cells to and from the container. As described above, the circulation path can comprise a 3D printed blood vessel bed comprising in order of cell flow an artery-scaled vessel, an arteriole-scaled vessel, a capillary-scaled vessel, a venule-scaled vessel, and a vein-scaled vessel. Next, at 504, a constant portion of the T-cells flows through the blood vessel bed for activation, transfecting/transducing and expanding the cells while flowing. Next, at 506, at least one of the activated, transfected, or expanded T-cells in the container is withdrawn. As noted above, during the method 500, the system controller 116 can be used to control one or more components of the system 100 and/or measure one or more variables associated with the system 100. For example, the system controller 116 can control a flow rate of the fluid through the circulation path, a temperature of the fluid in the container 104, etc.

While the foregoing is directed to embodiments of the present disclosure, other and further embodiments of the disclosure may be devised without departing from the basic scope thereof.

1. An apparatus for T-cell activation, T-cell transfection, and T-cell expansion, comprising:
a pump connected to a circulation path and configured to circulate cells suspended in a fluid to and from a container connected to the circulation path, the circulation path comprising a 3D printed blood vessel bed comprising in order of cell flow an artery-scaled vessel, an arteriole-scaled vessel, a capillary-scaled vessel, a venule-scaled vessel, and a vein-scaled vessel.

2. The apparatus of claim 1, wherein the artery-scaled vessel is connected to the circulation path and has an inner diameter from about 1 mm to about 2 cm, wherein the arteriole-scaled vessel is connected to the artery-scaled vessel and has an inner diameter from about 20 micron to about 1 mm, wherein the capillary-scaled vessel is connected to the arteriole-scaled vessel and has an inner diameter from about 5 micron to about 20 micron, wherein the venule-scaled vessel is connected to the capillary-scaled vessel and has an inner diameter from about 5 micron to about 20 micron, and wherein the vein-scaled vessel is connected to the venule-scaled vessel and has an inner diameter from about 1 mm to about 2 cm.

3. The apparatus of claim 1, wherein the 3D printed blood vessel bed is a single use 3D printed blood vessel bed and is made from at least one of polymeric material, biopolymer, hydrogel, or cells combined with hydrogels.

4. The apparatus of claim 1, wherein an interior of the artery-scaled vessel, the arteriole-scaled vessel, the capillary-scaled vessel, the venule-scaled vessel, and the vein-scaled vessel are at least one of smooth without any ridges or corrugations or porous.

5. The apparatus of claim 1, further comprising at least one electrode that is connected to at least one of the artery-scaled vessel, the arteriole-scaled vessel, the capillary-scaled vessel, the venule-scaled vessel, or the vein-scaled vessel and configured to generate an electrical impulse at the at least one of the artery-scaled vessel, the arteriole-scaled vessel, the capillary-scaled vessel, the venule-scaled vessel, or the vein-scaled vessel.

6. The apparatus of claim 1, wherein the container includes multiple ports each of which are configured to connect to corresponding one a gas supply, connect to a waste line, connect to a cell suspension apparatus, connect to a reagent apparatus, or connect to a media apparatus or in line monitoring devices and sensors.

7. The apparatus of claim 1, wherein the container comprises a temperature sensor that is configured to control a temperature of fluid within the container.

8. The apparatus of claim 1, further comprising a smart sensor that is configured to at least one of measure a glucose, lactate, glutamine, glutamate, pH, CO₂ or dissolved O level in the fluid, a pressure within the circulation path, a velocity of fluid flow through the circulation path, or a temperature of the fluid within the circulation path.

9. The apparatus of claim 1, wherein the 3D printed blood vessel bed is connected in line with the circulation path via an appropriate connector or via welding in a sterile welding operation.

10. A method for point of care treatment of a patient, comprising:
sterilely receiving patient T-cells into a container connected to a circulation path and a pump that is configured circulate the patient T-cells to and from the container, the circulation path comprising a 3D printed blood vessel bed comprising in order of cell flow an artery-scaled vessel, an arteriole-scaled vessel, a capillary-scaled vessel, a venule-scaled vessel, and a vein-scaled vessel;
expanding the patient T-cells while flowing a constant portion of the patient T-cells through the 3D printed blood vessel bed; and
sterilely withdrawing expanded patient T-cells in the container for administration back into the patient.

11. The method of claim 10, wherein the artery-scaled vessel is connected to the circulation path and has an inner diameter from about 1 mm to about 2 cm, wherein the arteriole-scaled vessel is connected to the artery-scaled vessel and has an inner diameter from about 20 micron to about 1 mm, wherein the capillary-scaled vessel is connected to the arteriole-scaled vessel and has an inner diameter from about 5 micron to about 20 micron, wherein the venule-scaled vessel is connected to the capillary-scaled vessel and has an inner diameter from about 5 micron to about 20 micron, and wherein the vein-scaled vessel is connected to the venule-scaled vessel and has an inner diameter from about 1 mm to about 2 cm.

12. The method of claim 10, wherein the 3D printed blood vessel bed is a single use 3D printed blood vessel bed and is made from at least one of polymeric material, biopolymer, hydrogel, or cells combined with hydrogels.

13. The method of claim 10, wherein an interior of the artery-scaled vessel, the arteriole-scaled vessel, the capillary-scaled vessel, the venule-scaled vessel, and the vein-scaled vessel are at least one of smooth without any ridges or corrugations or porous.

14. The method of claim 10, further comprising generating an electrical impulse at the at least one of the artery-scaled vessel, the arteriole-scaled vessel, the capillary-scaled vessel, the venule-scaled vessel, or the vein-scaled vessel.

15. The method of claim 10, wherein the container includes multiple ports each of which are configured to connect to corresponding one a gas supply, connect to a waste line, connect to a cell suspension apparatus, connect to a reagent apparatus, or connect to a media apparatus.

16. The method of claim 10, further comprising controlling a temperature of fluid within the container.

17. The method of claim 10, further comprising measuring at least one of a glucose, lactate, glutamine, glutamate, pH, CO₂ or dissolved O level in a fluid in the circulation path, a pressure within the circulation path, a velocity of fluid flow through the circulation path, or a temperature of the fluid within the circulation path.

18. The method of claim 10, wherein the 3D printed blood vessel bed is connected in line with the circulation path via an appropriate connector via welding in a sterile welding operation.

19. A method of activation, transfection, and expansion of T-cells, comprising:
receiving T-cells into a container connected to a circulation path and a pump that is configured circulate the T-cells to and from the container, the circulation path comprising a single use 3D printed blood vessel bed comprising in order of cell flow an artery-scaled vessel, an arteriole-scaled vessel, a capillary-scaled vessel, a venule-scaled vessel, and a vein-scaled vessel;
flowing a constant portion of the T-cells through the single use 3D printed blood vessel bed; and
withdrawing at least one of activated, transfected, or expanded T-cells in the container.

20. The method of claim 19, wherein the artery-scaled vessel is connected to the circulation path and has an inner diameter from about 1 mm to about 2 cm, wherein the arteriole-scaled vessel is connected to the artery-scaled vessel and has an inner diameter from about 20 micron to about 1 mm, wherein the capillary-scaled vessel is connected to the arteriole-scaled vessel and has an inner diameter from about 5 micron to about 20 micron, wherein the venule-scaled vessel is connected to the capillary-scaled vessel and has an inner diameter from about 5 micron to about 20 micron, and wherein the vein-scaled vessel is connected to the venule-scaled vessel and has an inner diameter from about 1 mm to about 2 cm.
